(19) United States
(12) Patent Application Publication
(10) Pub. No.: US 2022/0039907 A1
(43) Pub. Date: Feb. 10, 2022

(54) Inhibiting Post-Biopsy Inflammation and Metastasis

(71) Applicant: The Board of Regents of the University of Oklahoma, Norman, OK (US)

(72) Inventor: Takemi Tanaka, Edmond, OK (US)

(21) Appl. No.: 17/444,705

(22) Filed: Aug. 9, 2021

Related U.S. Application Data
(60) Provisional application No. 63/062,729, filed on Aug. 7, 2020.

(51) Int. Cl. A61B 90/00; A61L 31/14; A61L 31/16; A61L 31/04

(52) U.S. Cl. CPC A61B 90/39; A61L 31/145; A61L 31/16; A61L 2300/60; A61B 2090/3908; A61L 2300/41; A61L 31/04

(57) ABSTRACT

A method of inhibiting cancer cell metastasis in a subject by providing a biopsy marker device constructed of a polymeric material, the polymeric material containing an anti-inflammatory agent which is releasable over an extended period of time from the polymeric material after the biopsy marker device has been implanted in a tissue of the subject, and implanting the biopsy marker device into a tissue cavity of the subject, wherein the tissue cavity is caused by a biopsy performed on a tumor in the subject, and wherein release of the anti-inflammatory agent from the polymeric material does not begin for at least 18 to 24 hours after implantation and continues for at least 14 to 60 days after implantation. Metastasis of cancer cells from the tissue cavity is inhibited when the tumor is cancerous. The tissue may be, for example, breast, lung, prostate, pancreas, liver, kidney, uterus, ovary, intestine, stomach, or neck tissue.

### CROSS REFERENCE TO RELATED APPLICATIONS AND INCORPORATION BY REFERENCE STATEMENT

The present patent application claims priority under 37 CFR § 119(e) to United States Provisional Patent Application U.S. Ser. No. 63/062,729, filed on Aug. 7, 2020, the entire contents of which are hereby expressly incorporated herein by reference.

### BACKGROUND

Breast cancer (BC) is the most commonly diagnosed malignancy among women in the U.S., with over 268,600 expected new diagnoses in 2019 alone. Of those, over 80% are diagnosed at an early stage (Stage I-IIIA), yet recurrence rates remain as high as 13-30%. Despite improvements in 5-year survival, women with early-stage BC still face a significant risk of recurrence, metastatic progression, and death from breast cancer. Distant recurrence after successful initial surgical resection of the tumor suggests early phase systemic spread prior to surgery. Many factors contribute to metastatic progression; however, key aspects of the mechanisms underlying early phase systemic spread remain largely unknown. Treatments to inhibit the metastasis of breast cancer cells from the primary tumor site would be highly desirable.

### BRIEF DESCRIPTION OF THE DRAWINGS

The patent or application file contains at least one drawing executed in color. Copies of this patent with color drawing(s) will be provided by the Patent and Trademark Office upon request and payment of necessary fee.

FIG. 1 shows that needle biopsy induces organ metastasis in 4T1 BC mouse model (A); and shows representative luminescent images of mice with or without biopsy (B).

FIG. 2 shows the effect of time to tumor resection from biopsy on organ dissemination of py2T BC cells in biopsied mice and time matched control mice with no biopsy (n=8 per group).

FIG. 3 (A) shows that mortality risk steeply increases 35 days after biopsy; (B) likelihood of tumor size and node status upstaging by time to surgery; (C) distribution of interval between biopsy and surgery, over 30% of women wait for surgery >35 days; and (D) time to surgery steadily increases every year and minority women are affected more as the median time to surgery has already exceeded a 35 days window.

FIG. 4 shows (A) an H&E image of post-biopsy day 38 breast tumor with retention of fibrin matrix and its surrounding wound stroma at post-biopsy site; (B) close-up image of yellow dot area, carcinoma proximal to biopsy wound (yellow arrows point to BC cells with mesenchymal morphology); (C) close-up image of green dot area, wound stroma of biopsy wound; and (D) carcinomas distant from biopsy wound.

FIG. 5 shows micrograph images of STRO1+ MSCs (brown) in the peripheral stroma (A) and biopsy wound (B) of post-biopsy day 42 T1NOMO tumor; (C) SSEA4+ MSCs in biopsy wound in post-biopsy 15 days 4T1 murine tumor (inset represents tumor without biopsy); accumulation of GFP+MSCs (green) in the biopsy wound (D); and scattered infiltration to peripheral stroma in tumor without biopsy (E). Black ink in inset shows needle track.

FIG. 6 shows PGE₂ levels in biopsy-wound fluid (A), tumor (B), and blood (C).

FIG. 7 shows that intravenous injection of PGE₂ promoted dissemination of py230 BC cells to the lungs.

FIG. 8 is an illustration showing an overview model of biopsy-induced metastasis.

FIG. 9 shows the increase of py230 BC cells' dissemination to the lung in mice pre-treated with biopsy-WF (A) and the increase of pulmonary vessel permeability in mice treated with PGE₂ (B). (C) shows the infiltration of SSEA+ MSCs (red) to PGE₂-induced hyper-permeation sites (green). Blue: DAPI. Final mag at x40.

FIG. 10 shows that PGE₂ induced VCAM1 expression in human and mouse EC cell lines (A); PGE₂ induced VCAM1 expression in vessel surface of mouse lung (B); PGE₂ enhanced MSC adhesion to HMVECs (C); infiltration of GFP+ MSCs to the lung in PGE₂-treated mice. Green, GFP+ MSCs; Blue, DAPI. Final mag×200 (D); and activation of VLA4 of biopsy-WF treated MSCs (E). Increased FITC-LDV bindings to MSCs treated with biopsy-WF (green), compared to saline (red) or biopsy-WF+EDTA treated MSCs (blue).

FIG. 11 shows the inhibition of PGE₂-induced VCAM1 upregulation by EP4 selective inhibitor.

FIG. 12 shows results of metastasis free survival of mice with or without biopsy treated with Ibuprofen (A); and representative images of COX2 expression in the animals of the treatment (B).

FIG. 13 is a micrograph image of a biopsy wound with high COX2 expression. Brown, COX2; Blue, hematoxylin. X200.

FIG. 14 shows that oral administration of ibuprofen significantly reduced the PEG₂ level in biopsied mouse tumors to a level equal to the unbiopsied tumor (upper panel) and inhibition of biopsy-WF induced PGE₂ release from MSCs by the NSAIDs indomethacin, ibuprofen, and celecoxib (lower panel).

FIG. 15 shows micrographs of cavities left after biopsy removal of tumor tissue were treated with hydrogels containing celecoxib (left panel), or remained untreated (right panel). Micrographs are of biopsy cavities stained with H&E stain. Bar=100 μm.

FIG. 16 shows that in mice with breast cancer, biopsy of tumors enhanced metastasis of cancer cells to the lungs.

FIG. 17 shows immunohistochemistry micrographs of lung and tumor following biopsy.

FIG. 18 shows immunofluorescence (IF) micrographs of human breast tumor proximal to (upper panel) and distant from (lower panel) the biopsy site.

FIG. 19 shows that PGE₂ secretion increases following biopsy in mouse tumors.

FIG. 20 shows that COX2 activation is sustained following tumor biopsy.

FIG. 21 is a micrograph which shows accumulation of M2ϕ around a biopsy site in human breast tumors.

FIG. 22 shows that M2 polarization of Mϕ is enhanced in biopsied mouse tumors.

FIG. 23 shows immunohistochemical staining refults that further validated that F4/80+ Mϕ) and CD206+M2ϕ) were 7- and 35-fold higher around the needle track of the biopsy site of mouse tumors, respectively, compared to tumors distant from the needle tract of the biopsy site.

FIG. 24 shows Quantitative PCR results demonstrating an increase in M2 marker mRNA expression (arginase, YM1, YM2) and decrease in M1 marker mRNAs (iNOS and TNFα) in bone marrow-derived primary Mϕ incubated with abundant (10 μM) PGE2.

FIG. 25 shows that PGE₂-induced M2 polarization is prostaglandin E2 receptor (EP2) dependent.

FIG. 26 shows that biopsy-induced metastasis is inhibited in COX2 knock-out (KO) mice.

FIG. 27 shows that oral administration of the pharmacologic inhibitor for the COX2/EP2 axis blocked biopsy-induced metastasis.

FIG. 28 is an illustration showing a mechanism by which pharmacologic blockade of the PGE₂/EP2 signaling axis will reduce biopsy-induced metastasis.

### DETAILED DESCRIPTION

In certain non-limiting embodiments, the present disclosure is directed to biopsy markers which are coated with a drug-containing material, or contained in a drug-containing material, or which contain a drug-containing material. The drug is generally an anti-inflammatory drug. The drug-containing material may be a hydrogel, and the anti-inflammatory drug is generally released from the material over an extended period of time (e.g., for at least one week to 8 weeks). The material is formulated to delay release of the anti-inflammatory drug, in at least certain embodiments, for at least 18 to 24 hours after implantation at the biopsy site, such as within the biopsy cavity. In certain non-limiting embodiments, the present disclosure is directed to methods of reducing inflammation in tissue at a biopsy site. In certain non-limiting embodiments, the present disclosure is directed to methods of inhibiting metastasis of cancer cells from a biopsy site by reducing inflammation in tissue at a biopsy site. For example the cancer may be breast cancer, or any cancer in which there is a primary tumor which can be biopsied.

One non-limiting embodiment of the present disclosure is a method of inhibiting cancer cell metastasis in a subject, by providing a biopsy marker device which comprises a polymeric material which contains an anti-inflammatory agent which is releasable over an extended period of time from the polymeric material after the biopsy marker device has been implanted in a tissue of the subject; and implanting the biopsy marker device into a tissue cavity of the subject, wherein the tissue cavity is caused by a biopsy performed on a tumor in the subject, and wherein release of the anti-inflammatory agent from the polymeric material does not begin for at least 18 to 24 hours after implantation and continues for at least 14 to 60 days after implantation, wherein metastasis of cancer cells from the tissue cavity is inhibited when the tumor is cancerous. The biopsy marker device may be left implanted in the tissue cavity of the subject for at least 35 days. The polymeric material which contains the anti-inflammatory agent may be a hydrogel. The anti-inflammatory agent may be a nonsteroidal anti-inflammatory drug (NSAID). The anti-inflammatory agent may be a Cyclooxygenase-1 and/or Cyclooxygenase-2 inhibitor. The tissue of the subject in which the tumor is located may be, for example, breast, lung, prostate, pancreas, liver, kidney, uterus, ovary, intestine, stomach, or neck tissue.

Before further describing various embodiments of the present disclosure in more detail by way of exemplary description, examples, and results, it is to be understood that the compounds, compositions, and methods of present disclosure are not limited in application to the details of specific embodiments and examples as set forth in the following description. The description provided herein is intended for purposes of illustration only and is not intended to be construed in a limiting sense. As such, the language used herein is intended to be given the broadest possible scope and meaning; and the embodiments and examples are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the present disclosure. However, it will be apparent to a person having ordinary skill in the art that the present disclosure may be practiced without these specific details. In other instances, features which are well known to persons of ordinary skill in the art have not been described in detail to avoid unnecessary complication of the description. It is intended that all alternatives, substitutions, modifications, and equivalents apparent to those having ordinary skill in the art are included within the scope of the present disclosure. Thus, while the compounds, compositions, and methods of the present disclosure have been described in terms of particular (but non-limiting) embodiments, it will be apparent to those of skill in the art that variations may be applied to the compounds, compositions, and methods, and in the steps or in the sequence of steps of the methods described herein without departing from the concept, spirit, and scope of the inventive concepts.

All patents, patent applications, and non-patent publications including published articles mentioned in the specification or referenced in any portion of this application are hereby expressly incorporated by reference in their entirety to the same extent as if each individual patent or publication was specifically and individually indicated to be incorporated by reference.

Unless otherwise defined herein, scientific and technical terms used in connection with the present disclosure shall have the meanings that are commonly understood by those having ordinary skill in the art. Further, unless otherwise required by context, singular terms shall include pluralities, and plural terms shall include the singular. Where used herein, the specific term “single” is limited to only “one.”

As utilized in accordance with the methods, compounds, and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example. Reference to an integer with more (greater) or less than includes any number greater or less than the reference number, respectively. Thus, for example, reference to less than 100 includes 99, 98, 97, etc. all the way down to the number one (1); and less than 10 includes 9, 8, 7, etc. all the way down to the number one (1).

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” and “approximately” are used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The term “about” or “approximately,” where used herein when referring to a measurable value such as an amount, a temporal duration, and the like, is meant to encompass, for example, variations of ±20% or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment. Further, all references to one or more embodiments or examples are for purposes of illustration only and are to be construed as non-limiting of the claims.

The term “pharmaceutically acceptable” refers to compounds and compositions which are suitable for administration to humans and/or animals without undue adverse side effects such as (but not limited to) toxicity, irritation, and/or allergic response commensurate with a reasonable benefit/risk ratio. The compounds or conjugates of the present disclosure may be combined with one or more pharmaceutically-acceptable excipients, including carriers, vehicles, and diluents which may improve solubility, deliverability, dispersion, stability, and/or conformational integrity of the compounds or conjugates thereof.

By “biologically active” is meant the ability to modify the physiological system of an organism without reference to how the active agent has its physiological effects.

As used herein, “pure” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

Non-limiting examples of animals or subjects within the scope and meaning of these terms include dogs, cats, rats, mice, guinea pigs, chinchillas, horses, goats, cattle, sheep, zoo animals, Old and New World monkeys, non-human primates, and humans.

As used herein, the term “biodegradable” refers to the ability of a polymeric material to erode or degrade in vivo to form smaller chemical fragments. Degradation may occur, for example, by enzymatic, chemical or physical processes.

“Treatment” refers to therapeutic treatments. “Prevention” refers to prophylactic or preventative treatment measures or reducing the onset of a condition or disease. The term “treating” refers to administering the composition to a subject for therapeutic purposes and/or for prevention.

The terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art.

The term “effective amount” refers to an amount of an active agent which is sufficient to exhibit a detectable therapeutic or treatment effect in a subject without excessive adverse side effects (such as (but not limited to) substantial toxicity, irritation, and allergic response) commensurate with a reasonable benefit/risk ratio when used in the manner of the present disclosure. The effective amount for a subject will depend upon the subject's type, size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

The term “ameliorate” means a detectable or measurable improvement in a subject's condition, disease or symptom thereof. A detectable or measurable improvement includes a subjective or objective decrease, reduction, inhibition, suppression, limit or control in the occurrence, frequency, severity, progression, or duration of the condition or disease, or an improvement in a symptom or an underlying cause or a consequence of the disease, or a reversal of the disease. A successful treatment outcome can lead to a “therapeutic effect,” or “benefit” of ameliorating, decreasing, reducing, inhibiting, suppressing, limiting, controlling or preventing the occurrence, frequency, severity, progression, or duration of a disease or condition, or consequences of the disease or condition in a subject.

A decrease or reduction in worsening, such as stabilizing the condition or disease, is also a successful treatment outcome. A therapeutic benefit therefore need not be complete ablation or reversal of the disease or condition, or any one, most or all adverse symptoms, complications, consequences or underlying causes associated with the disease or condition. Thus, a satisfactory endpoint may be achieved when there is an incremental improvement such as a partial decrease, reduction, inhibition, suppression, limit, control or prevention in the occurrence, frequency, severity, progression, or duration, or inhibition or reversal of the condition or disease (e.g., stabilizing), over a short or long duration of time (hours, days, weeks, months, etc.). Effectiveness of a method or use, such as a treatment that provides a potential therapeutic benefit or improvement of a condition or disease, can be ascertained by various methods and testing assays.

In certain non-limiting embodiments, the drug-containing material is formulated to have a particular drug release profile, such that a certain amount of drug is released each day or over an extended period of multiple days or multiple weeks. For example such dosage of the active agent released in a subject could be in a range of 1 μg per kg of subject body mass to 1000 mg/kg, or in a range of 5 μg per kg to 500 mg/kg, or in a range of 10 μg per kg to 300 mg/kg, or in a range of 25 μg per kg to 250 mg/kg, or in a range of 50 μg per kg to 250 mg/kg, or in a range of 75 μg per kg to 250 mg/kg, or in a range of 100 μg per kg to 250 mg/kg, or in a range of 200 μg per kg to 250 mg/kg, or in a range of 300 μg per kg to 250 mg/kg, or in a range of 400 μg per kg to 250 mg/kg, or in a range of 500 μg per kg to 250 mg/kg, or in a range of 600 μg per kg to 250 mg/kg, or in a range of 700 μg per kg to 250 mg/kg, or in a range of 800 μg per kg to 250 mg/kg, or in a range of 900 μg per kg to 250 mg/kg, or in a range of 1 mg per kg to 200 mg/kg, or in a range of 1 mg per kg to 150 mg/kg, or in a range of 2 mg per kg to 100 mg/kg, or in a range of 5 mg per kg to 100 mg/kg, or in a range of 10 mg compound per kg to 100 mg/kg, or in a range of 25 mg per kg to 75 mg/kg.

Returning now to the description of particular embodiments, the present disclosure provides evidence that needle biopsy promotes distant metastasis in early stage breast cancer. Further provided are compositions and methods of treatment for reducing inflammation at the biopsy site and inhibiting metastasis of cancer cells therefrom. Evidence provided herein that needle biopsy promotes distant metastasis includes, (1) preclinical observations of increased organ metastasis incidence following biopsy of grafted tumors in two syngeneic mouse models of BC, (2) clinical observations of the appearance of breast cancer cells with invasive morphology adjacent to post-biopsy sites exhibiting chronic inflammation with accumulation of mesenchymal stem cells (MSCs), and (3) a bi-phasic increase in mortality risk associated with time from biopsy to surgery in patients with early-stage BC (n=210,533), with a 35 days lag-phase of no comparative increase in mortality risk followed by a distinct and accelerated increase in risk. Since BC takes years to develop, the 35-day lag-phase is not readily compatible with continuous metastatic seeding from tumors, but rather implies biopsy-triggered metastatic seeding. These observations indicate that needle biopsy followed by delayed surgery contributes to BC mortality. The clinical impact of the present disclosure is therefore extremely high given the number of women diagnosed and treated for early-stage BC each year. Since needle biopsy is an indispensable part of accurate diagnosis for the appropriate care of BC, compositions and methods disclosed herein for reducing inflammation in biopsy sites and metastasis therefrom will provide a post-biopsy therapeutic regimen that will improve survival for women with early-stage BC by reducing the mortality associated with metastatic breast cancer.

Without wishing to be bound by theory, it is hypothesized herein that release of prostaglandin E2 (PGE₂ or PGE2) from a chronically inflamed biopsy wound into the blood stream promotes pre-metastatic niche (PMN) formation through activation of the PGE₂ receptor type 4 (EP4)/VCAM1 axis on the endothelium of distant organs, coupled with the activation of α4131 integrin (VLA4), a ligand for VCAM1 of mesenchymal stem cells (MSCs). These collectively initiate PMN formation, permitting tissue dissemination of invasive BC cells. Therefore, pharmacologic blockade of PGE₂ biosynthesis, EP4/VCAM1 signaling, and VLA4 activation will prevent the initiation of the PMN and subsequent biopsy-associated BC metastasis.

There has been an alarming increase in time between biopsy and surgery for BC patients in the U.S., with >30% of early-stage BC patients delayed more 35 days to receive surgery between 2004 to 2014. Disconcertingly, median time to surgery increased annually from 23 to 32 days between 2004 to 2015, at a rate of 1 day each year. If this rate continues, it is estimated that the median time to surgery may already have reached 35 days, leaving an estimated 37,000 women diagnosed in 2019 at risk of surgical delay-associated mortality. While all patients were affected by the annual increase in time to surgery, minority women were over-represented among patients who experienced surgical wait times over 35 days. However, despite the scope of this problem, clinical awareness, as well as a biologic explanation for the necessity of timely surgery after diagnosis, has been severely lacking. Reflecting this knowledge gap, there are currently no guidelines for timely surgery or a post-biopsy therapeutic regimen in the U.S., allowing a continued upward drift in the interval between biopsy and surgery, thus, exposing an increasingly larger number of women in the U.S with early-stage BC to an avoidable increase in mortality risk. The safety of needle biopsy itself was granted by initial safety studies conducted in the 1960s to 1980s that compared patients receiving one-step, excisional biopsies to patients having an interval between needle biopsy and surgery of on average 18-25 days, which is well within the risk-free phase of the present work. While these old studies gave rise to the current clinical dogma that needle biopsy is safe and harmless, their applicability in the current medical setting is highly questionable, especially in an era when the interval between biopsy and surgery has continued to lengthen.

Despite the wound-inducing nature of needle biopsy, no standard post-biopsy medication is currently recommended. Rather, the major public patient education websites (ACS, Susan Komen, and NCI) recommend the use of an ice pad or “over the counter” pain killers such as Tylenol. Use of Non-steroidal anti-inflammatory drugs (NSAIDs) is recommended against due to concern over their blood-thinning effects which may cause bruising. While prompt surgery following diagnostic biopsy is ideal, achieving such a task may be a challenge given the racial/ethnic and socioeconomic disparities in access to care noted elsewhere herein. Thus, it is important to implement a clinically amenable, affordable, and safe solution for patients at risk of surgery delay.

In the past decade, biopsy markers have been implemented as a standard in clinical care. Biopsy markers are a radio-enhancing material that is inserted into the biopsy cavity after tumor tissue removal at the time of biopsy to help the clinician identify the position of the tumor at the time of surgery. HydroMARK® (Mammotome) has been broadly adopted as a new form of biopsy marker with a radio-enhancing titanium clip coated in a biocompatible/biodegradable hydrogel polymer composed of poly (lactic-co-glycolic acid) [PLGA]. HydroMARK is utilized for over 60% of patients undergoing breast biopsy at the inventor's institution. Hydrogel in the biopsy cavity enlarges by absorbing moisture and helps retention of the titanium biopsy marker within the cavity. A representative image of surgically resected Stage I breast tumor that received HydroMARK at the time of biopsy showed residual hydrogel remaining within the biopsy cavity after 50 days. Thus, a hydrogel can be used as a platform for the delayed and sustained local release of anti-inflammatory therapeutic payload within the biopsy cavity in accordance with the presently disclosed methods. In alternate embodiments, the payload holding material of the biopsy marker may comprise a porous material or sponge or a woven or non-woven fabric material, polymer, collagen, or any other implantable material known in the art which can be used for a sustained release of a therapeutic payload at a tissue site or other biological site.

Examples of biopsy markers (also referred to herein as tissue markers or biopsy site markers) and/or payload holding materials that can be used in the embodiments of the present disclosure include, but are not limited to, the biopsy marker devices and/or payload holding materials shown in U.S. Pat. Nos. 6,862,470; 9,486,431; and U.S. Patent Application Publication Nos. 2005/0142161; 2005/0143656; 2005/0234336; 2006/0036165; 2006/0079770; 2010/0324416; 2014/0094698; 2014/0194741; 2015/0157418; 2016/0120510; 2016/0151124; 2017/0066162; 2018/0221000; 2018/0353738; 2019/0090977; and 2019/0282325. Examples of hydrogels that can or have been used with biopsy markers include U.S. Pat. Nos. 6,083,524; 6,162,241; 6,270,464; 6,356,782; 6,605,294; 8,600,481; and 8,939,910, and U.S. Patent Application Publication 2017/0066162 and 2019/0090977. All of these U.S. patents and U.S. Patent Application Publications are incorporated herein by reference in their entirety.

In certain embodiments of the methods and/or biopsy marker devices provided herein, the material of the device which contains the payload (i.e., the drug or active agent) comprises a polymer. In some embodiments, the polymer comprises at least one of polyalkyl methacrylates, polyalkylene-co-vinyl acetates, polyalkylenes, polyurethanes, polyanhydrides, aliphatic polycarbonates, polyhydroxyalkanoates, silicone containing polymers, polyalkyl siloxanes, aliphatic polyesters, polyglycolides, polylactides, polylactide-co-glycolides, poly(e-caprolactone)s, polytetrahalooalkylenes, polystyrenes, poly(phosphasones), copolymers thereof, and combinations thereof.

In some embodiments of the methods and/or devices provided herein, the material comprises at least one bioabsorbable polymer, for example selected from: Polylactides (PLA); PLGA (poly(lactide-co-glycolide)); Polyanhydrides; Polyorthoesters; Poly(N-(2-hydroxypropyl) methacrylamide); DLPLA—poly(dl-lactide); LPLA—poly(l-lactide); PGA—polyglycolide; PDO—poly(dioxanone); PGA-TMC—poly(glycolide-co-trimethylene carbonate); PGA-LPLA—poly(l-lactide-co-glycolide); PGA-DLPLA—poly(dl-lactide-co-glycolide); LPLA-DLPLA—poly(l-lactide-co-dl-lactide); and PDO-PGA-TMC—poly(glycolide-co-trimethylene carbonate-co-dioxanone), and combinations, copolymers, and derivatives thereof. In some embodiments, the bioabsorbable polymer comprises between 1% and 95% glycolic acid content PLGA-based polymer.

In some embodiments of the methods and/or devices provided herein, the material comprises at least one of polycarboxylic acids, cellulosic polymers, proteins, polypeptides, polyvinylpyrrolidone, maleic anhydride polymers, polyamides, polyvinyl alcohols, polyethylene oxides, glycosaminoglycans, polysaccharides, polyesters, aliphatic polyesters, polyurethanes, polystyrenes, copolymers, silicones, silicone containing polymers, polyalkyl siloxanes, polyorthoesters, polyanhydrides, copolymers of vinyl monomers, polycarbonates, polyethylenes, polypropytenes, polylactic acids, polylactides, polyglycolic acids, polyglycolides, polylactide-co-glycolides, polycaprolactones, poly(e-caprolactone)s, polyhydroxybutyrate valerates, polyacrylamides, polyethers, polyurethane dispersions, polyacrylates, acrylic latex dispersions, polyacrylic acid, polyalkyl methacrylates, polyalkylene-co-vinyl acetates, polyalkylenes, aliphatic polycarbonates polyhydroxyalkanoates, polytetrahalooalkylenes, poly(phosphasones), polytetrahalooalkylenes, poly(phosphasones), and mixtures, combinations, and copolymers thereof. The polymers may be natural or synthetic in origin, including gelatin, chitosan, dextrin, cyclodextrin, Poly(urethanes), Poly(siloxanes) or silicones, Poly(acrylates) such as [rho]oly(methyl methacrylate), poly(butyl methacrylate), and Poly(2-hydroxy ethyl methacrylate), Poly(vinyl alcohol) Poly(olefins) such as poly(ethylene), [rho]oly(isoprene), halogenated polymers such as Poly(tetrafluoroethylene), and derivatives and copolymers such as those commonly sold as Teflon™ products, Poly(vinylidine fluoride), Poly(vinyl acetate), Poly(vinyl pyrrolidone), Poly(acrylic acid), Polyacrylamide, Poly(ethylene-co-vinyl acetate), Poly(ethylene glycol), Poly(propylene glycol), Poly(methacrylic acid); etc. Suitable polymers also include absorbable and/or resorbable polymers including the following, combinations, copolymers and derivatives of the following: Polylactides (PLA), Polyglycolides (PGA), PolyLactide-co-glycolides (PLGA), Polyanhydrides, Polyorthoesters, Poly(N-(2-hydroxypropyl) methacrylamide), Poly(l-aspartamide), including the derivatives DLPLA—poly(dl-lactide); LPLA—poly(l-lactide); PDO—poly(dioxanone); PGA-TMC—poly(glycolide-co-trimethylene carbonate); PGA-LPLA—poly(l-lactide-co-glycolide); PGA-DLPLA—poly(dl-lactide-co-glycolide); LPLA-DLPLA—poly(l-lactide-co-dl-lactide); and PDO-PGA-TMC—poly(glycolide-co-trimethylene carbonate-co-dioxanone), and combinations thereof. In some embodiments, the polymer is capable of becoming soft after implantation, for example by hydration, degradation or both.

In some embodiments of the methods and/or devices provided herein, the bioabsorbable polymer is resorbed within at least one of: about 5 days, about 7 days, about 14 days, about 3 weeks, about 4 weeks, about 30 days, about 35 days, about 38 days, about 45 days, about 60 days, about 90 days, about 180 days, about 6 months, about 9 months, or about 1 year.

In some embodiments of the methods and/or devices provided herein, the bioabsorbable polymer is resorbed within a range of: about 5 days to about 2 weeks, about 5 days to about 3 weeks, about 5 days to about 4 weeks, about 5 days to about 6 weeks, about 1 week to about 4 weeks, about 2 weeks to 5 weeks, about 3 weeks to about 6 weeks, about 3 weeks to about 8 weeks, about 4 weeks to about 8 weeks, about 30 days to about 60 days, about 30 days to about 90 days, about 60 to about 90 days, about 90 to about 180 days, about 60 days to about 180 days, about 180 days to about 365 days, about 2 months to about 6 months, about 4 months to about 8 months, or about 6 months to about 12 months.

In some embodiments of the methods and/or devices provided herein, the material comprises a biodegradable material that is adhered and/or cohered, coated to, or contained in the biopsy device. The biodegradable material is capable of degrading over time. In some embodiments, the pharmaceutical agent and/or the active agent is released from the material within at least one of about 12 hours, about 14 hours, about 16 hours, about 18 hours, about 24 hours, about 36 hours, about 48 hours, about 3 days, about 4 days, about 5 days, about 7 days, about 14 days, about 3 weeks, about 4 weeks, about 30 days, about 35 days, about 38 days, about 40 days, about 45 days, about 60 days, about 90 days, about 180 days, about 6 months, about 9 months, or about 1 year. Non-limiting examples of biodegradable polymeric materials that can be used to form a biodegradable polymer material for use herein can include polyester, polyamide, polycarbonates, polyanhydrides, polyorthoesters, polycaprolactone, polyesteramides, polycyanoacrylate, polyetherester, poly(phosphates), poly(phosphonates), poly(phosphites), polyhydric alcohol esters, blends and copolymers thereof.

In certain embodiments, the release of the anti-inflammatory agent from the polymeric material continues, after implantation of the biopsy marker device, for at least 10 days, or at least 11 days, or at least 12 days, or at least 13 days, or at least 14 days, or at least 15 days, or at least 16 days, or at least 17 days, or at least 18 days, or at least 19 days, or at least 20 days, or at least 21 days, or at least 22 days, or at least 23 days, or at least 24 days, or at least 25 days, or at least 26 days, or at least 27 days, or at least 28 days, or at least 29 days, or at least 30 days, or at least 31 days, or at least 32 days, or at least 33 days, or at least 34 days, or at least 35 days, or at least 36 days, or at least 37 days, or at least 38 days, or at least 39 days, or at least 40 days, or at least 41 days, or at least 42 days, or at least 43 days, or at least 44 days, or at least 45 days, or at least 46 days, or at least 47 days, or at least 48 days, or at least 49 days, or at least 50 days, or at least 51 days, or at least 52 days, or at least 53 days, or at least 54 days, or at least 55 days, or at least 56 days, or at least 57 days, or at least 58 days, or at least 59 days, or at least 60 days, or any range which combines the values of the boundaries of any of said periods, such as, for example, from 14 days to 56 days, from 14 days to 48 days, from 21 to 56 days, or 21 days to 42 days.

In some embodiments of the methods and/or devices provided herein, the material comprises a microstructure. In some embodiments, particles of the active agent are sequestered or encapsulated within said microstructure. In some embodiments, the microstructure comprises microchannels, micropores and/or microcavities. In some embodiments, the microstructure is selected to allow sustained release of the active agent. In some embodiments, the microstructure is selected to allow controlled release of the active agent. Given the number of women who are affected by prolonged time to surgery, the clinical impact of the present disclosure is high with an immediate clinically translatable prevention strategy to implement a safe and low-cost therapeutic regimen when timely surgery is unfeasible.

In certain non-limiting embodiments, the drug-containing material is a hydrogel comprising a thermosensitive PLGA-PEG-PLGA tri-block copolymer. The PLGA-PEG-PLGA tri-block copolymer (e.g., purchased from Sigma Aldrich (Cat no: 908843)) contains molecular weights (1600-1500-1600) with a lactide:glycolide ratio 75:25. One gr of PLGA-PEG-PLGA viscous pellet was combined with 6 ml of ice-cold PBS and allowed to stir for 24 h at 4° C. for dissolving and for forming a homogeneous solution. The 1 gram in 6 ml PBS solution becomes 16.66% of W/V ratio. It is aliquoted into small tubes and stored at 4° C. The aliquots exhibit a solution state at 4° C. and at room temperature, and solidifies when incubated at 37° C. The copolymer solution is then mixed with an anti-inflammatory drug such as, but not limited to, Celecoxib (e.g., from Sigma Aldrich (Cat no. PHR1683).

In one non-limiting embodiment, the PLGA-PEG-PLGA tri-block copolymer solution is loaded with Celecoxib by dissolving 25 mg of Celecoxib in 50 μl of DMSO, then combining that mixture with 800 μl of PLGA-PEG-PLGA co-polymer solution (16%), which is then allowed to stir for 24 at 4° C. for loading and to get a homogeneous solution. After forming the homogeneous Celecoxib-loaded thermogel solution, the mixture is incubated at 37° C. for 2-5 mins wherein it solidifies. This can then be combined with the biopsy marker device, then implanted into a biopsy site.

To estimate the amount/concentration of drug released from the hydrogel, the released drug is collected in PBS solutions according to the following procedure. Three ml of PBS with 0.2% Tween 20 is added to the mixture as a drug eluting medium. This solution is shaken at 50 rpm at 37° C. at pre-determined intervals. The liquid PBS can be collected and the same amount of fresh PBS added. Released drug can be measured by HPLC or other spectrophotometric methods. The solutions can be stored at −80° C.

In certain embodiments, the anti-inflammatory agent used in the formulations described herein for inhibiting biopsy-induced metastasis may be a non-selective NSAID (such as but not limited to diclofenac, diflunisal, etodolac, fenoprofen, flurbiprofen, ibuprofen, indomethacin, ketoprofen, ketorolac, mefenamic acid, meloxicam, nabumetone, naproxen, oxaprozin, piroxicam, sulindac, and tolmetin); a COX2-selective NSAID (such as but not limited to celecoxib, rofecoxib, and valdecoxib); an EP2 selective antagonist (such as but not limited to PF04418948, K(B) 2-20, TG6-10-1, TG8-4, TG8-21, and other “TG” class compounds shown in U.S. Pat. Nos. 9,518,044, 10,040,783, and 10,052,332, and in the tables in Ganesh, T., J. Jiang, and R. Dingledine, “Development of second generation EP2 antagonists with high selectivity” Eur. J. Med Chem. 2014 Jul. 23; 82:521-535; an EP4 selective antagonist (such as but not limited to CJ-023423, LY3127760, RQ-15986, AAT-008, E7046 (ER0886046), CR6086, L-161,982, and ONO-4578); or an anti-α4 (anti-VLA-4) integrin monoclonal antibody (such as but not limited to Natalizumab, Vedolizumab, LS-C152894, R1-2, and GG5/3 Ab)). Non-limiting dosages that can be provided to inhibit metastasis include, but are not limited to: 5, 30, or 50 mg of CJ-023423 per kg BW, 40, 200, or 500 mg of celecoxib per kg BW, and 10, 100, or 300 μg of Natalizumab, or other dosages noted elsewhere herein.

### Experimental

Data provided herein show that diagnostic needle biopsy leaves a chronic wound in the tumor and systemically promotes organ metastasis. A post-biopsy wound remained in the breast tumor for a prolonged period, exhibiting abundant bone marrow-derived MSCs coupled with high levels of PGE₂ in the tumor and blood stream that induced systemic changes conducive to metastasis. Consistent with these results, epidemiologic evidence shows a sharp and continued linear increase in mortality risk following the first 35 days between biopsy and surgery during which no excess mortality risk accumulated for patients with early-stage BC (n=210,533). This observed 35-day lag-phase in mortality risk increase is not readily explained by continuous metastatic seeding from tumors, but rather implies biopsy-induced metastatic seeding. These observations point to needle biopsy as contributing to BC mortality.

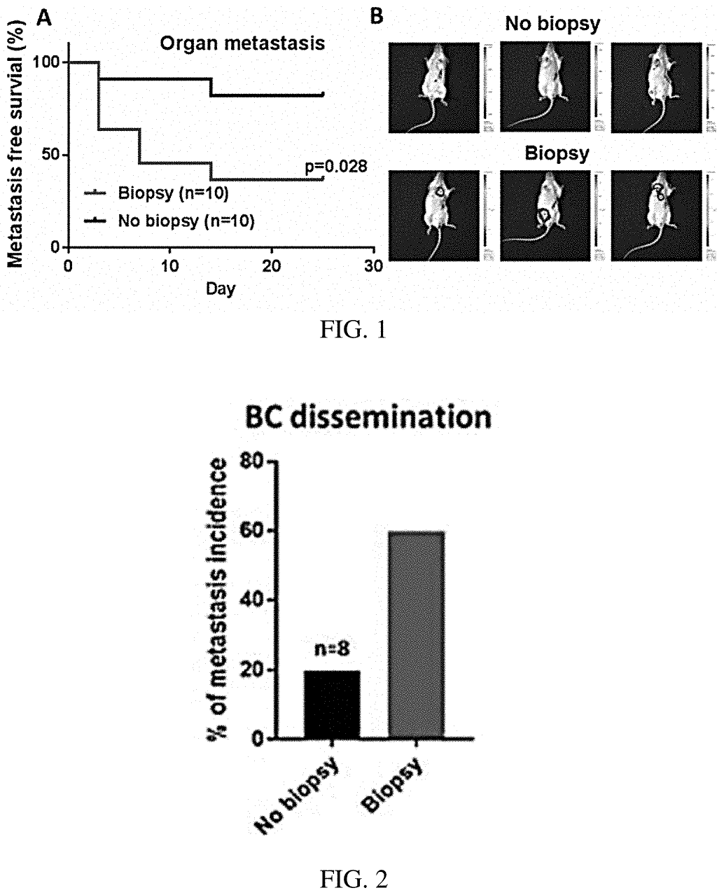

A needle biopsy collects a fraction of a tumor and is an essential, non-optional procedure for definitive diagnosis of BC. As shown herein, needle biopsy of breast tumors resulted in increased incidence of metastasis in two independent syngeneic BC mouse models using two different metastasis detection methods (bioluminescence and genomic DNA analysis). Detection of measurable organ metastasis using bioluminescence showed that needle biopsy causes increased and earlier onset of distant metastases in the 4T1 murine BC model (FIG. 1). In the py2T murine BC model, BC dissemination to the lung occurred more frequently among biopsied mice compared to mice without biopsy, and longer retention of the biopsied tumor without surgical resection led to a drastic increase in organ dissemination of BC (FIG. 2). Biopsy, however, had no major effect on tumor growth in both models (not shown).

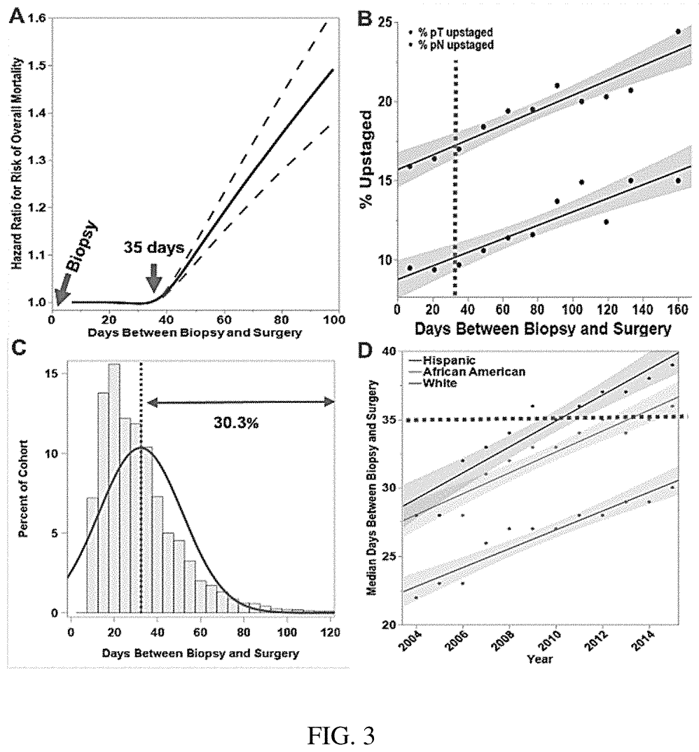

To explore the hypothesis of whether needle biopsy induces pro-metastatic changes and contributes to eventual mortality, a cohort study was performed to evaluate the effect of time between biopsy and surgery on survival among early-stage BC patients (NCDB, n=210,533) who received surgery as their first treatment between 2004 and 2014. After multivariable adjustment for other factors (age, race, tumor characteristics, poverty, education, comorbidity, chemotherapy, endocrine therapy, radiotherapy, and so on), risk of mortality remained constant for the first 35 days, but then cumulatively increased by approximately 5-6% every two weeks compared to the initial risk free period (FIG. 3(A)). If increased mortality simply reflected natural disease progression, the hazard ratio would be expected to rise continually from the time of diagnostic biopsy (day 0) and not display an initial 35-day lag-period (FIG. 3(A)). This highlights the possibility that needle biopsy instigates pro-metastatic changes that lead to BC mortality. Similar to the change in mortality risk, the likelihood of pathologic upstaging (either enlargement of tumor size or node negative to positive transition) increased with the duration between biopsy to surgery in early stage BC patients (FIG. 3(B)), suggesting that BC progresses with time to surgery from biopsy. Along the same lines, a randomized clinical study suggested higher mortality among women who received needle biopsy compared to excisional biopsy (Hansen NM, Ye X, Grube BJ, Giuliano AE. Manipulation of the primary breast tumor and the incidence of sentinel node metastases from invasive breast cancer. Arch Surg. 2004; 139(6):634-639; discussion 639-640). Also, the frequency of patients waiting more than 35 days from biopsy to receive surgery was high, approximately a third of the cohort (FIG. 3(C)). Disconcertingly, it was further observed that median time to surgery increased steadily from 23 to 32 days between 2004 and 2015, at a rate of approximately 1 day each year (FIG. 3(D)). While all races were affected by the annual increase in time to surgery, minority and low socioeconomic status women were over-represented among patients who experienced surgery wait times over about 38 days (pink line in FIG. 3(D)). Currently, the time required for Medicaid enrollment/approval for surgery after diagnosis is 2-6 weeks (depending on the state of residence), causing a major bottleneck for timely surgery. Although necessary, increased use of second opinions, multimodal imaging, molecular profiling, and other precision medicine initiatives slow decision making and also contribute to the growing problem of surgical delay for early stage BC. However, clinical awareness of the potential risk posed by the retention of the biopsied tumor for a prolonged time is severely lacking. This critical knowledge gap has allowed a steady upward drift in the interval between biopsy and surgery, exposing a large number of women with early-stage BC to an avoidable increase in mortality risk. Accordingly, in 2015 (the most updated data available in NCDB), as many as 41% of the women (n=11,364) did not receive surgery before 35 days post-biopsy, 57% of Hispanic, 51% of African American, and 38.6% of White women were affected. Reflecting this gap in knowledge, there are currently no guidelines for timely surgery or a post-biopsy therapeutic regimen in the U.S.

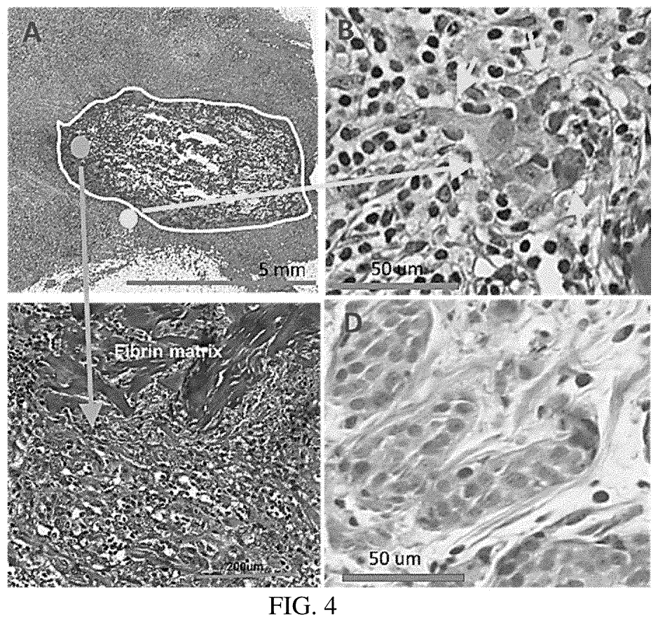

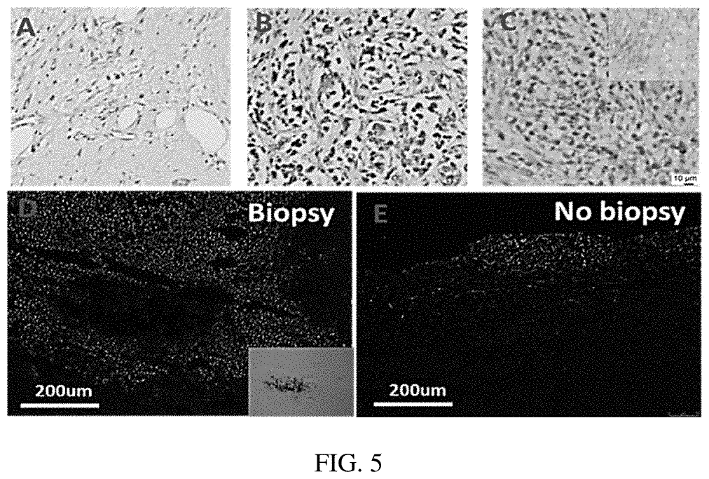

In seeking an explanation for biopsy-associated metastasis, it was further discovered that needle biopsy consistently leaves an unhealed wound with continuous inflammation in clinical BC tumors. A representative image of Stage I, node-negative breast tumors surgically removed 38 days after biopsy shows persistent high-cellularity wound stroma with the fibrin matrix surrounded by inflammatory cells (FIG. 4(A), white circle). Histologic analysis of clinical samples of early-stage BC (22 cases) with time from biopsy to surgery ranging from 30-57 days showed that nearly ˜70% of cases exhibit retention of a chronic wound around the needle tract within the tumors. Disconcertingly, adjoining isolated or clusters of cancer cells displayed mesenchymal-like invasive morphology with visible protrusions indicated by yellow arrows (FIG. 4(B)). The arrangement of a biopsy wound where BC cells readily interface stromal components within scanty ECM is distinct from the naturally developed tumor stroma (FIG. 4(C)), where low cellularity stromal cells are tightly embedded within the ECM and partitioned from BC cells (FIG. 4(D)). While the typical wound repair process completes within 2 weeks, by which time the fibrin matrix and infiltrating cells are cleared, the biopsy wound was characterized by an abundance of bone marrow-derived mesenchymal stem cells (MSCs), STRO-1⁺ in humans (FIGS. 5(A-B)) and SSEA4⁺ in mice (FIG. 5(C)). In an experimental mouse model, adoptive transfer of GFP+ bone marrow-derived MSCs showed preferential accumulation in biopsy wounds (FIG. 5(D)), whereas infiltration of GFP+ MSCs was far less and limited only to the peripheral stroma in unbiopsied tumors (FIG. 5(E).

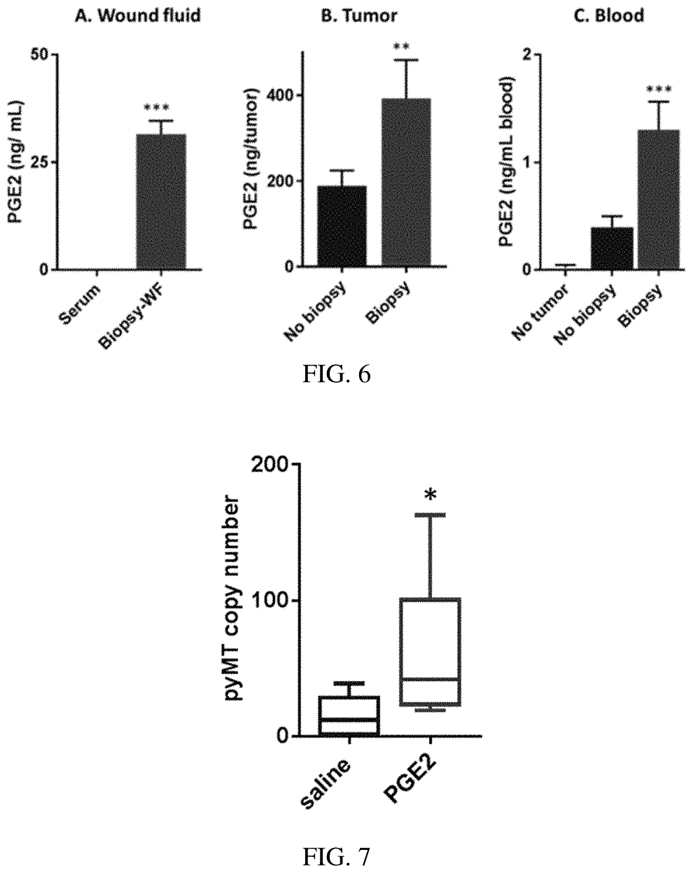

Wounding triggers a repair cascade by forming a fibrin matrix containing a series of chemo-attractants (e.g., TGF-β, bFGF, PDGF, TNFα, and EGF) that recruit MSCs from bone marrow to the injured site. MSCs produce innumerable growth factors, cyto-chemokines, and metabolites that promote angiogenesis, immunomodulation, and ECM remodeling in order to repair the damaged tissue. MSCs are suggested to polarize into two distinct phenotypes: pro-inflammatory (MSC1) or anti-inflammatory (MSC2) with Th1 or Th2-like functions, respectively. MSC2 is characterized by a high level of PGE₂ release, a potent vasodilater. Reflecting the abundance of MSCs in biopsied tumors, PGE₂ levels were significantly higher in wound fluid (WF) collected from biopsied mouse tumors than serum isolated from unbiopsied mice (FIG. 6(A)), and biopsied tumors than unbiopsied ones (FIG. 6(B)). Given the high interstitial pressure coupled with insufficient lymphatic drainage due to superficial lymphatic vessels in solid tumors, soluble factors seep into the circulation from the tumor. Accordingly, PGE₂ levels in the blood were 3 times higher in the biopsied mice compared to that in mice bearing unbiopsied tumors or mice without tumor burden (FIG. 6(C)). PGE₂ is enzymatically converted from arachidonic acid by the rate-limiting enzyme Cyclooxygenase (COX). COX2 expression is induced by inflammatory stimuli, whereas COX1 is constitutive. Priming of mice with intravenous injection of PGE₂ (at a level equal to PGE₂ present in post-biopsy blood) followed by injection of py230 BC cells led to enhanced pulmonary metastasis compared to untreated mice (FIG. 7). These observations suggest that leakage of PGE₂ into the circulation from MSC enriched, unhealed biopsy wounds promotes metastasis, which may account for higher mortality among women with prolonged retention of the biopsy wound prior to definitive treatment (either surgery or chemotherapy). However, the biologic mechanism by which circulating PGE₂ promotes metastasis has heretofore remained unknown.

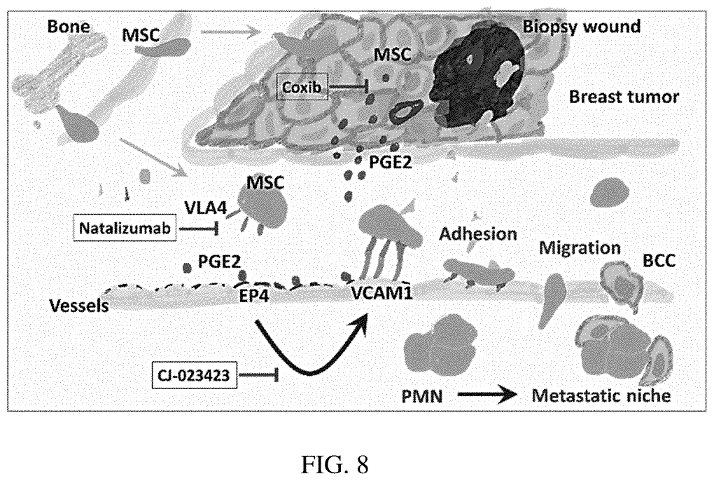

Without wishing to be bound by theory, it is believed that soluble factors that leak into the circulation from biopsied tumors promote metastasis. It is therefore proposed herein that circulating PGE₂ derived from MSCs in the chronically inflamed biopsy wound initiates pre-metastatic niche formation through activation of the EP4/VCAM1 axis in pulmonary vessels, where VLA4 (VLA-4) activated bone marrow-derived circulating MSCs bind. These simultaneous systemic changes, in turn, promote homing of MSCs to lung parenchyma and initiate pre-metastatic niche (PMN) formation and BC organ dissemination. Therefore, blockade of PGE₂ biosynthesis, VLA4 activation, or EP4 effectively inhibits biopsy-associated metastasis (FIG. 8).

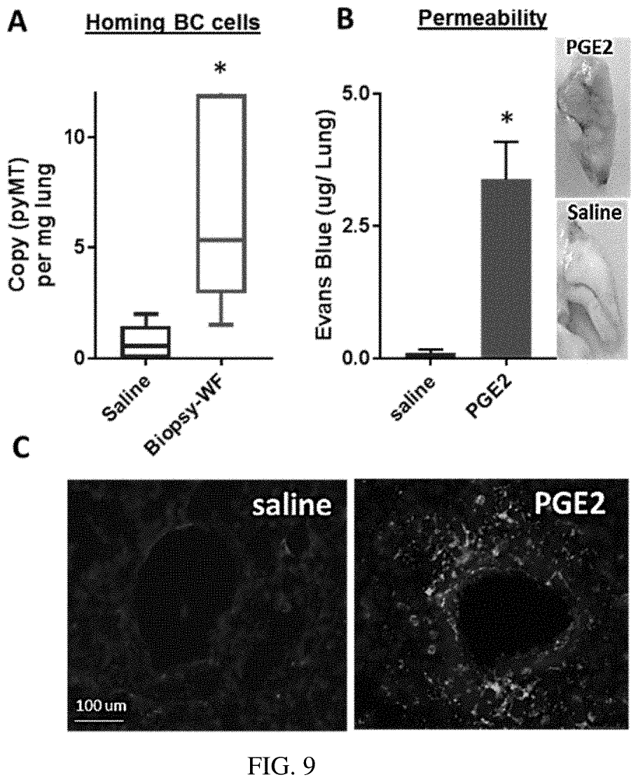

Wounding triggers a healing process through local secretion of an array of soluble factors that mediate tissue repair. Soluble factors also leak into the circulation from the wound and have been suggested to stimulate distant metastasis. It has been discovered herein that intravenous injection of biopsy-induced surgical wound fluids (WF) prior to BC cell injection promotes metastasis in mice (FIG. 9(A)).

Successful hematogenous metastasis requires two sequential independent extravasation steps for circulating cells to exit the blood stream and migrate into tissues. Tissue homing of bone marrow-derived mesenchymal stem cells (MSCs), and immune cells at distant organs leads to PMN formation, and cancer cell homing at the PMN gives rise to metastasis. The PMN is a metastasis conducive microenvironment that is pre-established before cancer cell arrival and is composed of a variety of circulating cells (MSCs and immune cells), fibroblasts, and inflamed endothelium. Tissue homing of circulating cells is governed by a multi-step adhesion cascade, shear resistant adhesion to the vessel wall, basal membrane degradation, followed by trans-endothelial migration. Vascular adhesion molecules (i.e., E-selectin, P-selectin, ICAM1, and VCAM1) on the endothelial surface mediate catch-bond of circulating cells down to the vessel wall under swift blood flow through affinity binding with their respective counter-receptor ligands. In the context of PMN formation, tumor-derived soluble factors (i.e., cyto-chemokines, exosome, and miRNA) mobilize MSCs from the bone marrow, prime hematopoietic cells, and activate the endothelium of distant organs.

For successful tissue homing of MSCs, VCAM1 engagement with its ligand, integrin α4β1 (VLA4) with active conformation is essential for their tissue infiltration. Thus, how soluble factors derived from biopsy-wound promote PMN formation and metastasis was investigated in the present work. We examined whether WF collected from biopsy wounds (referred to as biopsy-WF) promotes BC metastasis. Intravenous injection of tumor biopsy-WF prior to injection of py230 murine BC cells (3,000 cells) showed far more potent metastasis induction compared to control mice that received saline in an experimental metastasis model (FIG. 9), supporting the present hypothesis that soluble factors derived from the biopsy wound promote metastasis. Based on the continual abundance of MSCs in biopsied tumors, the involvement of PGE₂ was suspected since MSCs are a robust producer of PGE₂. As anticipated, PGE₂ levels in biopsied mouse tumors were found to be two times higher than that of the same size tumors without biopsy (FIG. 5(B)). Additionally, consistent with the high level of PGE₂ in biopsy-WF, PGE₂ levels in the plasma were 3 times higher in biopsied mice compared to mice without biopsy and mice with no tumor burden (FIG. 5(C)).

Finally, intravenous injection of PGE₂ at a level equal to the plasma in biopsied mice showed enhanced py230 BC cells' dissemination to the lungs in an experimental metastasis model (FIG. 6). Intravenous injection of PGE₂ caused increased vessel permeability shown by higher Evans Blue leakage into the lungs compared to saline-injected ones (FIG. 9(B)). PGE₂-induced hyper-permeation sites indicated by leakage of 40 kDa FITC-dextran (green) coincided with accumulation of SSEA4⁺ MSCs (red), a key component of the PMN⁵¹ (FIG. 9(C)). These data suggested PGE₂ is a potent inducer of PMN formation by enhancing tissue migration of bone marrow-derive MSCs into the lung parenchyma.

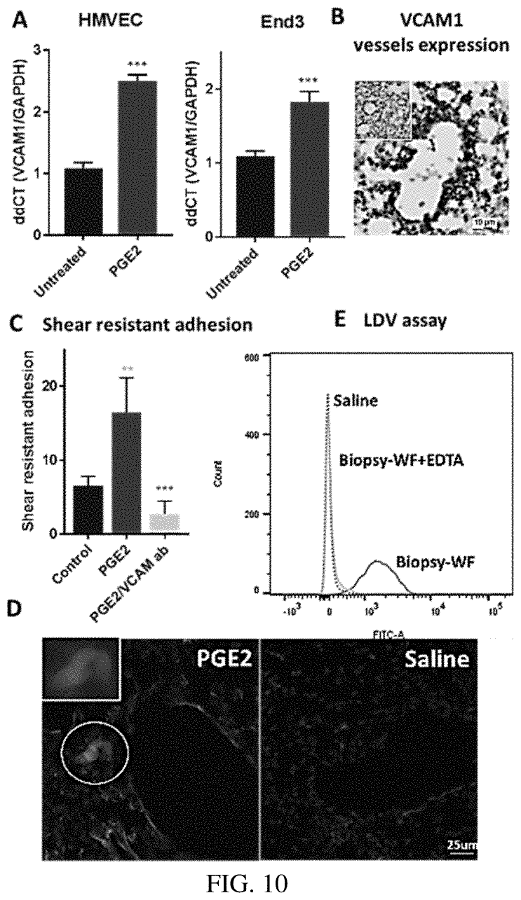

Inflamed vessels serve as a gateway for MSCs to enter tissue parenchyma. VCAM1 engagement to integrin α4β1 (very late antigen 4; VLA4) mediates shear resistant adhesion of MSCs to the vessel wall that leads to their trans-endothelial migration. While VCAM1 expression on the vessel wall is typically almost absent, incubation with a PGE₂ level equivalent to post-biopsy blood was found to induce VCAM1 mRNA expression in both human microvessel endothelial cells (HMVECs) and a murine endothelial cell line (End.3) (FIG. 10(A)). Similarly, VCAM1 vascular expression was elevated in PGE₂ treated mice compared to untreated ones (FIG. 10(B)). Accordingly, shear resistant adhesion of human MSCs was 2-fold higher to PGE₂ treated HMVECs than to untreated ones; however, VCAM1 neutralizing antibody abrogated PGE₂-induced shear resistant adhesion of MSCs (FIG. 10(C)). Additionally, adaptive transfer of freshly isolated GFP+ MSCs into PGE₂ pre-treated mice showed their preferential infiltration to perivascular space (FIG. 10(D)). The white circle in the left panel of FIG. 10(D) indicates infiltrating GFP+ MSCs, and the inset in the upper left corner is a close-up image of the corresponding location. Since VLA4 is the ligand for VCAM1, and conformational change of VLA4 is prerequisite for binding to VCAM1, ligand binding assay using FITC-LDV peptide (which selectively binds to an active form of VLA4) was performed. FACS analysis showed that FITC-LDV peptide bound avidly to biopsy-WF treated human MSCs, although the addition of EDTA (for measurement of non-specific binding) completely abrogated the binding (FIG. 10(E)), suggesting that tumor biopsy-derived soluble factors activate VLA4 of MSCs. Therefore, these data suggested that leakage of soluble factors from biopsy wounds may hold dual effects in activating both VCAM1 expression in the endothelium and VLA4 activation in MSCs, collectively, contributing to biopsy-induced PMN formation and metastasis.

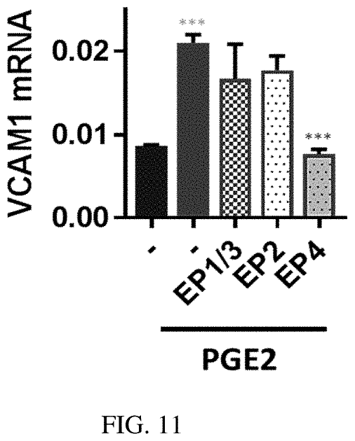

PGE₂ exerts its action through the binding to 4 types of prostaglandin E receptor (EP1, 2, 3, and 4), which ubiquitously express in various cell types. Human endothelial cells express all 4 types of EP receptors. Selective antagonist against EP4 (L-161982) blocked PGE₂-induced VCAM1 expression in End.3, while blockade of EP1/3 (0N08711) and EP2 (PF-04418948) showed no effect (FIG. 11). Therefore, without wishing to be bound by theory, an explanation of biopsy-induced metastasis is that systemic availability of PGE₂ leads to an elevation of VCAM1 expression in the pulmonary vessels through the EP4 receptor, resulting in increased MSCs tissue infiltration that initiates a provisional PMN in the perivascular space of the lungs (see overview model in FIG. 8).

As noted above, it is believed that soluble factor leakage from biopsied tumors into circulation initiates provisional PMN formation and subsequent metastasis. The binding of circulating PGE₂ to EP4 in pulmonary vascular endothelium stimulates the expression of VCAM1. In parallel, soluble factors in biopsy-WF activate α4β1 integrin (VLA4), enabling engagement with VCAM1, collectively promoting tissue homing of circulating VLA4 active MSCs to the perivascular space in the lungs to initiate pre-metastatic niche conducive to BC metastasis.

Tumor-derived soluble factors (e.g., miRNA, exosomes, metabolites, cytokines) have been reported as inducers of PMN formation. Data provided herein show that PGE₂ at a level equal to the plasma of post-biopsied mice promotes (1) lung metastasis (FIG. 6), (2) VCAM1 expression in endothelial cells (ECs) (FIG. 10(A)), (3) shear resistant adhesion of bone marrow-derived mesenchymal stem cells (MSCs) (FIG. 10(C)), and (4) infiltration of GFP+ MSCs into the perivascular space of the lungs (FIG. 10(D)). Based on the above evidence and abundance of PGE₂ in mice bearing biopsied tumors, it is believed that PGE₂ released from the chronically inflamed biopsy wound into the circulation induces pulmonary vessel changes conducive to metastasis.

Despite the wound-inducing nature of needle biopsy of breast tumors, no standard post-biopsy therapeutic regimen is currently recommended. Rather, the major public patient education websites (ACS, Susan Komen, and NCI) recommend the use of an ice pad or oral “over the counter” pain killers such as Tylenol. Non-steroidal anti-inflammatory drugs (NSAIDs) such as ibuprofen and coxibs are not recommended due to concern about their blood thinning effects (Martini WZ, Rodriguez CM, Deguzman R, et al. Dose Responses of Ibuprofen In Vitro on Platelet Aggregation and Coagulation in Human and Pig Blood Samples. Mil Med. 2016; 181(5 Suppl): 111-116). While the present mortality risk data indicate that receipt of timely surgery within 35 days of biopsy is important to maximize the survival benefit for patients with early-stage BC, it is also critical to implement a clinically amenable preventive solution for patients who for various reasons may not receive timely surgery. The present NCDB cohort analysis found significant racial/ethnic and socioeconomic disparities in timeliness of surgery (FIG. 3), which is concerning if the delay of surgery in part contributes to disproportionally high BC mortality among racial minorities and women of lower socioeconomic status. Timely surgery is ideal; but it requires coordinated efforts among the payers, providers, and patients. This further highlights the need for alternative solutions such as a post-biopsy medication to prevent biopsy-induced progressive pro-metastatic changes.

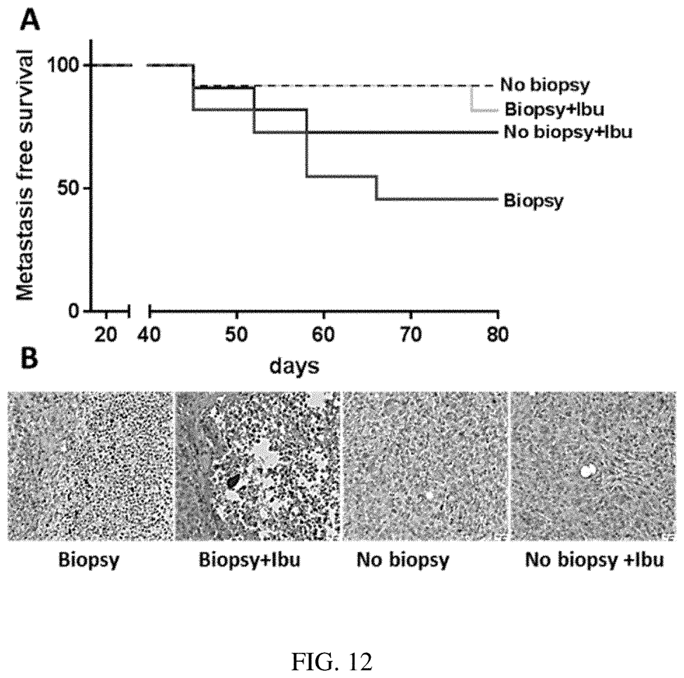

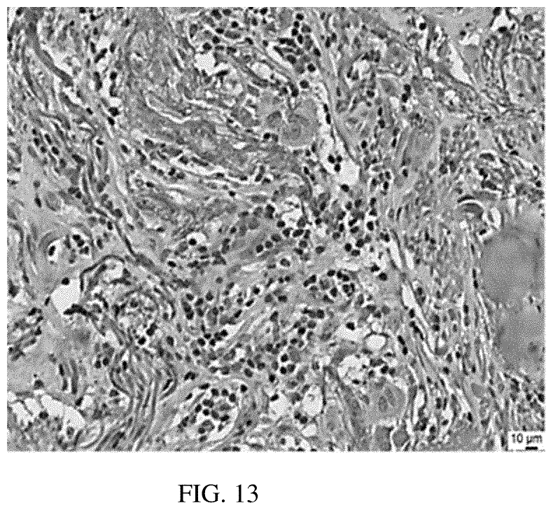

To experimentally explore the hypothesis that inhibition of PGE₂ biosynthesis by NSAIDs diminishes biopsy-induced metastasis, 4T1-Luc tumors were biopsied or left unbiopsied (n=10 per group) once tumor size reached 100 mm³. One day later, daily oral ibuprofen (non-selective COX inhibitor) or saline was started (50 mg/kg) for 9 days, until the day before mastectomy. Mice were then monitored for metastasis over the next 3 months. The Kaplan-Meier curve for organ metastasis-free survival showed that metastasis occurs most frequently in the biopsied group throughout the monitoring period (6/10 mice); however, oral administration of ibuprofen significantly reduced metastasis incidence (2/10 mice) and delayed onset (FIG. 12(A)). Ibuprofen had no significant effect on metastasis in mice that did not receive biopsy. Histopathologic evaluation of mastectomized tumors showed intense COX2 expression in a broad area of the biopsied wound with innumerable infiltrating cells. In contrast, cellularity and COX2 expression in the biopsy wound were far less in mice that were treated with ibuprofen (FIG. 12(B)). Similarly, analysis of Stage 1 breast tumor, surgically resected 42 days after biopsy showed high COX2 expression in the biopsy wound (FIG. 13).

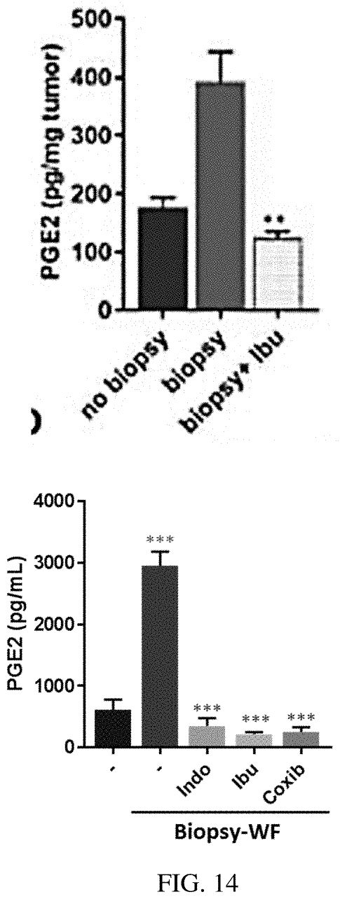

Since ibuprofen is a non-selective COX inhibitor that blocks both COX1 and COX2, it was further explored which COX is responsible for PGE₂ production from MSCs that are overabundant in the biopsy wound. Oral administration of ibuprofen significantly reduced the PEG₂ level in biopsied mouse tumors to a level equal to the unbiopsied tumor (FIG. 14, upper panel). Treatment of the primary mouse MSCs with biopsy-WF resulted in a burst PGE₂ release, and COX non-selective inhibitors (indomethacin and ibuprofen) and COX2 selective inhibitor (celecoxib) equally abrogated biopsy-WF induced PGE₂ release (FIG. 14, lower panel), further justifying the use COX2 selective inhibitor for prevention of biopsy-associated metastasis. A PLGA hydrogel containing celecoxib was injected into cavities formed by removal of biopsy tissue of tumors. Biopsied tumors that received the celecoxib-containing hydrogel were histologically less chaotic with a hollow biopsy cavity and reduced inflammatory cells in comparison to untreated biopsied tumors which had abundant inflammatory cells (FIG. 15).

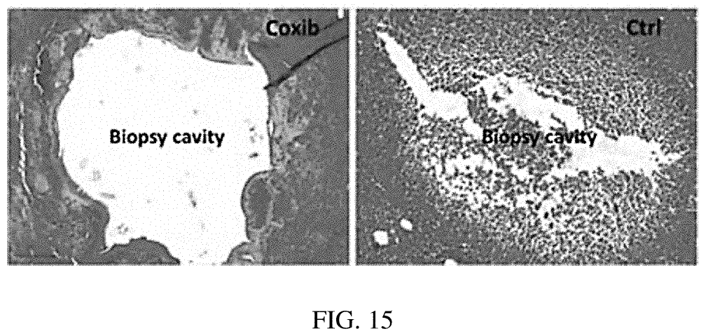

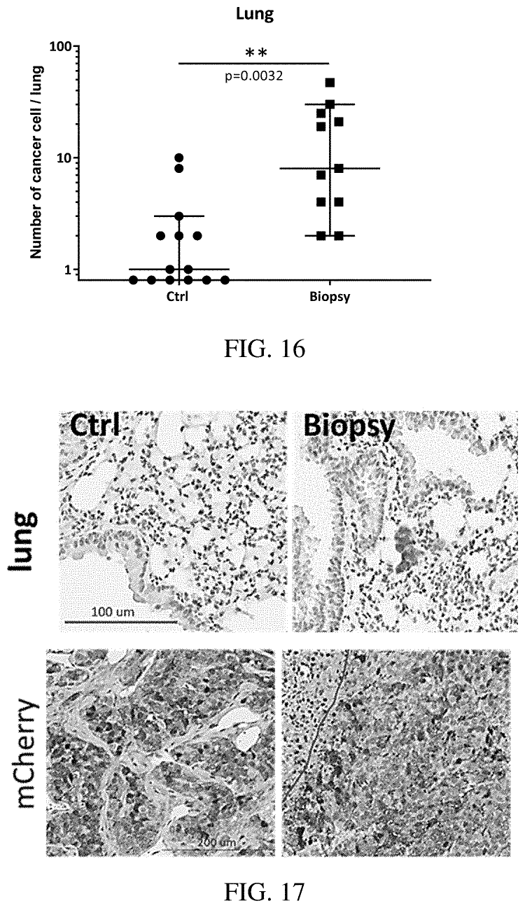

To evaluate whether needle biopsy promotes breast cancer metastasis, age-matched female mice (n=15), bored with an orthotopically implanted breast tumor that was derived from mCherry reporter gene (red) transfected py230 murine breast cancer cells, were needle biopsied once or left unbiopsied. FCS analysis of a single cell suspension of the whole lung collected 15 days following the tumor biopsy showed a significantly higher number of mCherry+ cells present in the lung than unbiopsied tumors (FIG. 16). Immunohistochemical staining for mCherry confirmed the presence of small metastatic lesions in the lung of biopsied mice. In parallel, mCherry+ cells (brown) displayed elongated mesenchymal-like morphology adjacent to the needle track, as indicated by red arrows, without a clear margin between cancer cells and stroma, indicating acquisition of invasiveness in cancer cells (i.e. epithelial to mesenchymal transition; FIG. 17). Multicolor immunofluorescence staining of human breast cancer cases further confirmed the EMT, displaying an increased expression of vimentin (green) in parallel with reduced expression of E-cadherin (red) in panCK+ (white) cancer cells adjacent to the needle track (top image; FIG. 18). In contrast, the absence of vimentin and intense E-cadherin expression in panCK+ cancer cells were seen distant from the needle tract in py230 tumors (bottom). Collectively, these data support that needle biopsy of breast tumors promotes EMT and subsequent lung metastasis.

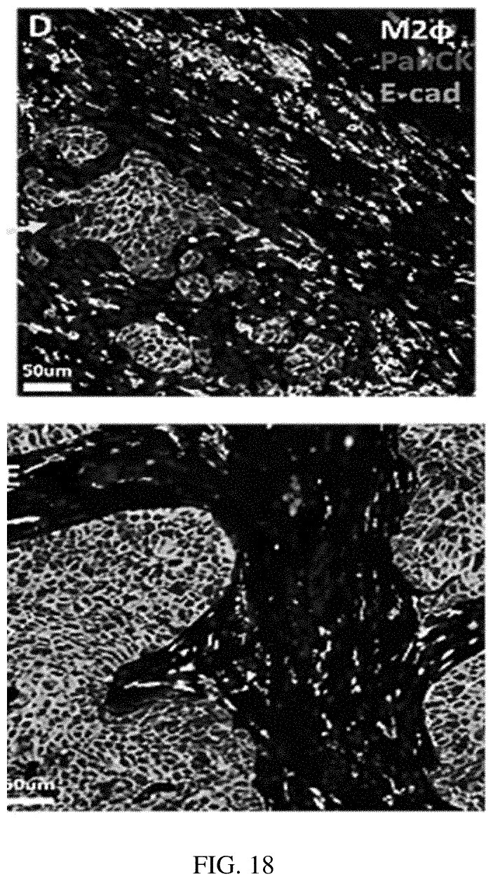

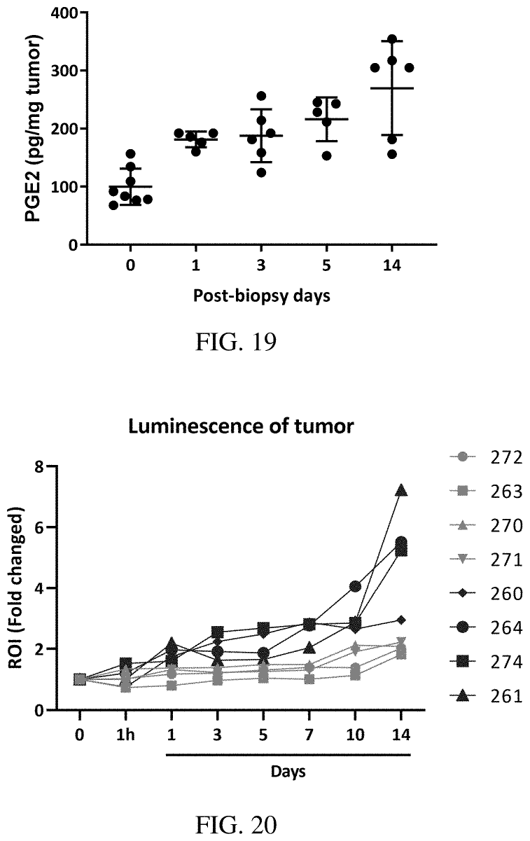

Based on the substantial retention of inflammatory cells at post-biopsy site, we examined the level of prostaglandin E2 (PGE₂), a mediator of inflammation, at various time points following biopsy of py230 tumors. PGE₂ levels were elevated 1 day after the biopsy and continued to increase until 14 days, compared to unbiopsied control tumors (day 0; FIG. 19). Additionally, in vivo COX2 promoter assay was performed. Lethally irradiated recipient mice (n=4) had an adoptive transfer of BMDCs, which were isolated from COX2^{Luc} donor mice and orthotopically injected with py230 cells. Then, their tumors were biopsied or left unbiopsied. Longitudinal non-invasive monitoring showed an initial rapid rise in COX2 promoter activity within an hour following tumor biopsy (red lines), followed by an exponential increase (FIG. 20). In contrast, no significant changes were seen in COX2 promoter activity in mice that did not receive a biopsy (grey line), indicating that tumor biopsy instigates COX2 promoter activity and subsequent PGE2 release from infiltrating bone marrow-derived cells.

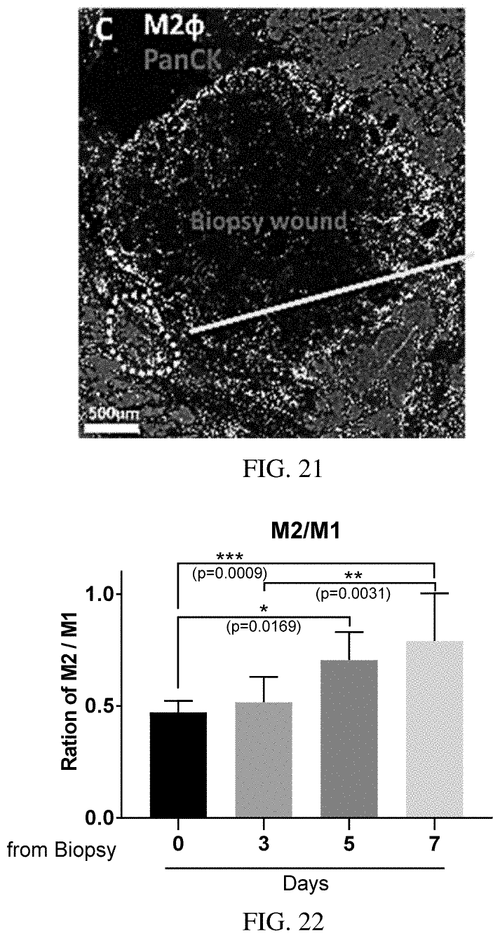

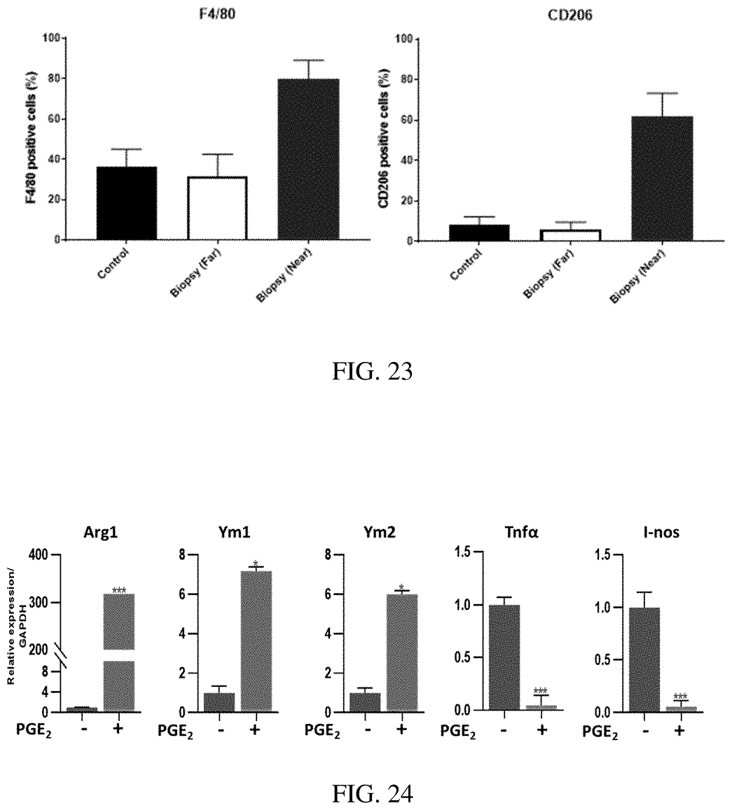

Double immunofluorescent images of human breast cancer cases showed a substantial accumulation of CD206+M2ϕ (white) around the needle track by forming a ring-like structure, interfacing to the adjacent panCK+ tumor (red fluorescence; FIG. 21). By contrast, there were less M2 Mϕ present in the peripheral stroma. Additional FACS analysis of py230 tumors, with various time points after biopsy, showed a significantly higher proportion of M2ϕ beginning at post-biopsy day 5 and following, compared to unbiopsied day 0 control tumors (n=7), while no difference was noted in the proportion of M1ϕ. Reflecting the disproportional increase of M2ϕ, M2/M1 ratio was significantly higher in biopsied tumors, suggesting biopsy triggers M2 shift (FIG. 22). Immunohistochemical staining further validated that F4/80+Mϕ and CD206+M2ϕ were 7- and 35-fold higher around the needle track, respectively, compared to tumors distant from the needle tract (FIG. 23).

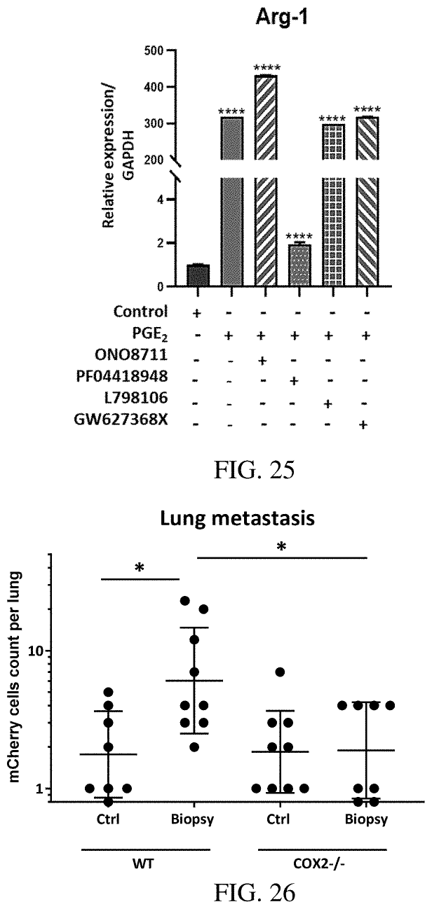

To further identify the biological consequences of abundant PGE₂, the bone marrow-derived primary Mϕ was incubated with 10 μM PGE2. Quantitative PCR showed an increase in M2 marker mRNA expression (arginase, YM1, YM2) and decrease in M1 marker mRNAs (iNOS and TNFα; FIG. 24). PGE₂ exerts its action through 4 integral membranous E prostanoid receptors (EP1, EP2, EP3, and EP4). The Mϕ was incubated with PGE₂ and selective pharmacologic inhibitors against respective prostaglandin E2 receptor (EP2). EP2 inhibitor (PF04418948) abrogated PGE₂-mediated arginase expression, while no inhibitory effect was observed by EP4 (L-161982), EP1 (0N08711), or EP3 (L-798106) inhibitors (FIG. 25), suggesting that PGE2-induced M2 shift is EP2-dependent. In seeking the biological consequence of sustained COX2 activation triggered by biopsy, we found that biopsy-induced py230^{mcherry} metastasis was diminished to a level similar to that of unbiopsied mice with BMDCs^{COX-/-} or BMDCs^{wt} transfer (FIG. 26). Thus, these data suggest that sustained COX2 activation in infiltrating BMDCs in response to biopsy triggers breast cancer metastasis.

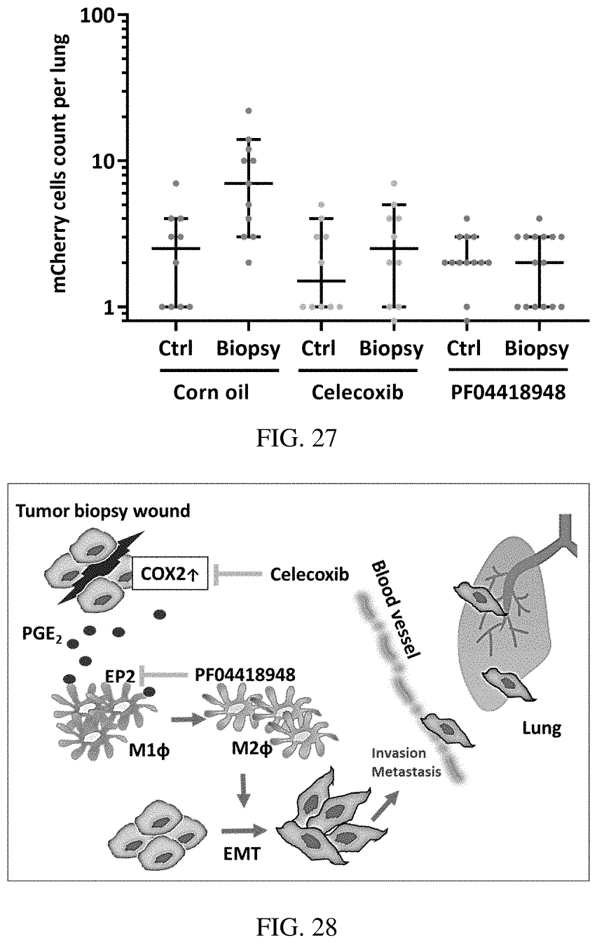

Given the role of COX2/EP2 axis in biopsy-induced metastasis, we postulated that pharmacologic inhibition of COX2 or EP2 would diminish biopsy-induced metastasis. Py230^{mCherry} tumor bearing mice (n=15 per group) were orally administered selective COX inhibitor (celecoxib) or EP2 inhibitor (PF04418948) daily following biopsy. Biopsy of tumor resulted in a lung metastasis; however, oral administration of celecoxib or PF04418948 significantly reduced biopsy-induced lung metastasis (FIG. 27). These data indicate that biopsy-induced metastasis is pharmacologically preventable or mitigatable. The mechanism of biopsy-induced metastasis is mediated through the abundance of PGE₂-producing BMDCs in the post-biopsy site, which skew neighboring Mϕ towards M2, promoting cancer cell invasion. Thus, pharmacologic blockade of the PGE₂/EP2 signaling axis will reduce biopsy-induced metastasis (FIG. 28).

While the present disclosure has been described herein in connection with certain embodiments so that aspects thereof may be more fully understood and appreciated, it is not intended that the present disclosure be limited to these particular embodiments. On the contrary, it is intended that all alternatives, modifications and equivalents are included within the scope of the present disclosure as defined herein. Thus the examples described above, which include particular embodiments, will serve to illustrate the practice of the inventive concepts of the present disclosure, it being understood that the particulars shown are by way of example and for purposes of illustrative discussion of particular embodiments only and are presented in the cause of providing what is believed to be the most useful and readily understood description of procedures as well as of the principles and conceptual aspects of the present disclosure. Changes may be made in the formulations of the various compositions described herein, the methods described herein or in the steps or the sequence of steps of the methods described herein without departing from the spirit and scope of the present disclosure. Further, while various embodiments of the present disclosure have been described in claims herein below, it is not intended that the present disclosure be limited to these particular claims.

What is claimed is:

1. A method of inhibiting cancer cell metastasis in a subject, comprising:
providing a biopsy marker device which comprises a polymeric material, the polymeric material containing an anti-inflammatory agent which is releasable over an extended period of time from the polymeric material after the biopsy marker device has been implanted in a tissue of the subject; and
implanting the biopsy marker device into a tissue cavity of the subject, wherein the tissue cavity is caused by a biopsy performed on a tumor in the subject, and wherein release of the anti-inflammatory agent from the polymeric material does not begin for at least 18 to 24 hours after implantation and continues for at least 14 to 60 days after implantation.

2. The method of claim 1, wherein metastasis of cancer cells from the tissue cavity is inhibited when the tumor is cancerous.

3. The method of claim 1, wherein the biopsy marker device is left implanted in the tissue cavity of the subject for at least 35 days.

4. The method of claim 1, wherein the polymeric material which contains the anti-inflammatory agent is a hydrogel.

5. The method of claim 1, wherein the anti-inflammatory agent is a nonsteroidal anti-inflammatory drug (NSAID).

6. The method of claim 1, wherein the anti-inflammatory agent is a Cyclooxygenase-1 and/or Cyclooxygenase-2 inhibitor.

7. The method of claim 1, wherein the tissue of the subject in which the tumor is located is selected from the group consisting of breast, lung, prostate, pancreas, liver, kidney, uterus, ovary, intestine, stomach, and neck.

8. A method of inhibiting cancer cell metastasis in a subject, comprising:
providing a biopsy marker device which comprises a polymeric material, the polymeric material containing a nonsteroidal anti-inflammatory drug (NSAID) which is releasable over an extended period of time from the polymeric material after the biopsy marker device has been implanted in a tissue of the subject; and
implanting the biopsy marker device into a tissue cavity of the subject, wherein the tissue cavity is caused by a biopsy performed on a tumor in the subject, and wherein release of the NSAID from the polymeric material does not begin for at least 18 to 24 hours after implantation and continues for at least 14 to 60 days after implantation.

9. The method of claim 8, wherein metastasis of cancer cells from the tissue cavity is inhibited when the tumor is cancerous.

10. The method of claim 8, wherein the biopsy marker device is left implanted in the tissue cavity of the subject for at least 35 days.

11. The method of claim 8, wherein the polymeric material which contains the anti-inflammatory agent is a hydrogel.

12. The method of claim 8, wherein the tissue of the subject in which the tumor is located is selected from the group consisting of breast, lung, prostate, pancreas, liver, kidney, uterus, ovary, intestine, stomach, and neck.

13. A method of inhibiting cancer cell metastasis in a subject, comprising:
providing a biopsy marker device which comprises a hydrogel, the hydrogel containing an anti-inflammatory agent which is releasable over an extended period of time from the hydrogel after the biopsy marker device has been implanted in a tissue of the subject; and
implanting the biopsy marker device into a tissue cavity of the subject, wherein the tissue cavity is caused by a biopsy performed on a tumor in the subject, and wherein release of the anti-inflammatory agent from the hydrogel does not begin for at least 18 to 24 hours after implantation and continues for at least 14 to 60 days after implantation, and wherein the biopsy marker device is left implanted in the tissue cavity of the subject for at least 35 days.

14. The method of claim 13, wherein metastasis of cancer cells from the tissue cavity is inhibited when the tumor is cancerous.

15. The method of claim 13, wherein the anti-inflammatory agent is a nonsteroidal anti-inflammatory drug (NSAID).

16. The method of claim 13, wherein the anti-inflammatory agent is a Cyclooxygenase-1 and/or Cyclooxygenase-2 inhibitor.

17. The method of claim 13, wherein the tissue of the subject in which the tumor is located is selected from the group consisting of breast, lung, prostate, pancreas, liver, kidney, uterus, ovary, intestine, stomach, and neck.
